(19) United States
(12) Patent Application Publication
(10) Pub. No.: US 2015/0307543 A1
(43) Pub. Date: Oct. 29, 2015

(54) POLYMORPH

(71) Applicant: ONCOLOGY RESEARCH INTERNATIONAL LIMITED, Perth, Western Australia (AU)

(72) Inventors: David Millar Walker, Wellington (NZ); Philip Andrew Marshall, Tennyson (AU)

(73) Assignee: ONCOLOGY RESEARCH INTERNATIONAL LIMITED, Perth, WA (AU)

(21) Appl. No.: 14/654,149

(22) PCT Filed: Apr. 24, 2013
(86) PCT No.: PCT/AU2013/000417

§ 371 Date: Jun. 19, 2015

(30) Foreign Application Priority Data
Dec. 21, 2012 (AU) 2012905703
Apr. 11, 2013 (AU) 2013203998

(51) Int. Cl. C07J 71/00

(52) U.S. Cl. CPC C07J 71/0005

(57) ABSTRACT

The present invention relates to a new polymorphic form of the compound diosgenyl α-L-rhamnopyranosyl-(1->2)-β-D-glucopyranoside (compound I) and pharmaceutical compositions containing this polymorph

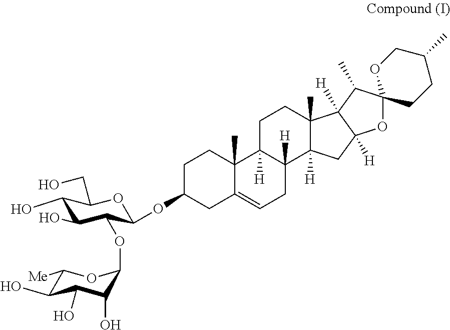

### FIELD

The present invention relates to a new polymorphic form of the compound diosgenyl α-L-rhamnopyranosyl-(1->2)-β-D-glucopyranoside and pharmaceutical compositions containing this polymorph.

### BACKGROUND

The compound diosgenyl α-L-rhamnopyranosyl-(1->2)-β-D-glucopyranoside (Compound I) is a known natural compound that occurs in trace amounts in a number of rare plant species. The compound shows significant promise as a pharmaceutically active agent for the treatment of a number of medical conditions and clinical development of this compound is underway based on the activity profiles demonstrated by the compound.

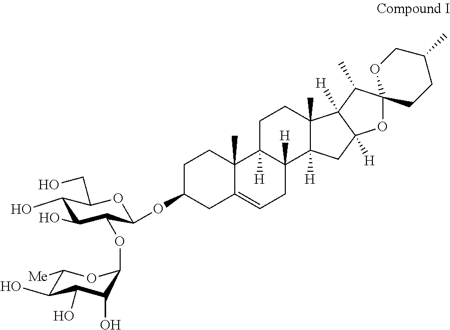

In the development of a drug suitable for mass production and ultimately commercial use acceptable levels of drug activity against the target of interest is only one of the important variables that must be considered. For example, in the formulation of pharmaceutical compositions it is imperative that the pharmaceutically active substance be in a form that can be reliably reproduced in a commercial manufacturing process and which is robust enough to withstand the conditions to which the pharmaceutically active substance is exposed.

In a manufacturing sense it is important that during commercial manufacture the manufacturing process of the pharmaceutically active substance be such that the same material is reproduced when the same manufacturing conditions are used. In addition it is desirable that the pharmaceutically active substance exists in a solid form where minor changes to the manufacturing conditions do not lead to major changes in the solid form of the pharmaceutically active substance produced. For example it is important that the manufacturing process produce material having the same crystalline properties on a reliable basis and also produce material having the same level of hydration.

In addition it is important that the pharmaceutically active substance be non-hygroscopic, stable both to degradation and subsequent changes to its solid form. This is important to facilitate the incorporation of the pharmaceutically active substance into pharmaceutical formulations. If the pharmaceutically active substance is hygroscopic (“sticky”) in the sense that it absorbs water (either slowly or over time) it is almost impossible to reliably formulate the pharmaceutically active substance into a drug as the amount of substance to be added to provide the same dosage will vary greatly depending upon the degree of hydration. Furthermore variations in hydration or solid form (“polymorphism”) can lead to changes in physico-chemical properties, such as solubility or dissolution rate, which can in turn lead to inconsistent oral absorption in a patient.

Finally depending upon the form that the compound is administered in the material handling properties of the compound must be taken into consideration. This includes such considerations such as the way in which the compound can flow (if in a powdered form) and how easily the compound is to dissolve in order to produce liquid formulations.

Accordingly, chemical stability, solid state stability, “shelf life” and materials handling properties (such as ease of solubilising the compound) of the pharmaceutically active substance are very important factors. In an ideal situation the pharmaceutically active substance and any compositions containing it, should be capable of being effectively stored over appreciable periods of time, without exhibiting a significant change in the physico-chemical characteristics of the active substance such as its activity, moisture content, solubility characteristics, solid form and the like. Furthermore in an ideal situation the compound should be readily able to be easily dissolved in suitable solvents in order to produce liquid formulations.

With any drug candidate there is a balance between these potentially competing properties. Nevertheless an important property of any drug is its stability and therefore it is desirable that the drug exhibit low hygroscopicity so that it can be reproducibly dosed. In circumstances where a drug is relatively hygroscopic it is found to absorb sufficient water that reproducible dosing and material handling is difficult.

As such it would be desirable to identify polymorphic forms of this compound that provide a better combination of properties in comparison to the known polymorph. As a result of their studies the present applicants have identified a polymorph that has significantly lower hygroscopicity than the known polymorphs while at the same time having a higher bulk density.

### SUMMARY

The present invention provides a crystalline form of a compound of the formula:

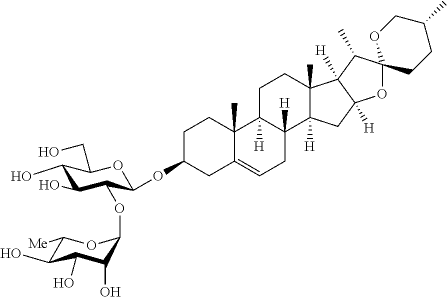

which shows on X-ray diffraction a peak on the 2theta scale at 2.96±0.02°.

In some embodiments the crystalline form also shows on X-ray diffraction at least 1 peak on the 2theta scale selected from the group consisting of 17.33°±0.02°, 17.43°±0.02°, 17.60°±0.02°, 19.84°±0.02°, and 20.03°±0.02°.

In some embodiments the crystalline form shows on X-ray diffraction at least 2 peaks on the 2theta scale selected from the group consisting of 17.33°±0.02°, 17.43°±0.02°, 17.60°±0.02°, 19.84°±0.02°, and 20.03°±0.02°.

In some embodiments the crystalline form shows on X-ray diffraction at least 3 peaks on the 2theta scale selected from the group consisting of 17.33°±0.02°, 17.43°±0.02°, 17.60°±0.02°, 19.84°±0.02°, and 20.03°±0.02°.

In some embodiments the crystalline form shows on X-ray diffraction at least 4 peaks on the 2theta scale selected from the group consisting of 17.33°±0.02°, 17.43°±0.02°, 17.60°±0.02°, 19.84°±0.02°, and 20.03°±0.02°.

In some embodiments the crystalline form shows on X-ray diffraction peaks on the 2theta scale at 17.33°±0.02°, 17.43°±0.02°, 17.60°±0.02°, 19.84°±0.02°, and 20.03°±0.02°.

In some embodiments the crystalline form shows on X-ray diffraction at least 1 peak on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

In some embodiments the crystalline form shows on X-ray diffraction at least 4 peaks on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

In some embodiments the crystalline form shows on X-ray diffraction at least 7 peaks on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

In some embodiments the crystalline form shows on X-ray diffraction peaks on the 2theta scale at 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

The present invention also provides a pharmaceutical composition comprising the crystalline form as described above.

### BRIEF DESCRIPTION OF THE DRAWINGS

FIG. 1 DSC of a polymorphic form of compound I isolated from methanol.

FIG. 2 DSC of the polymorphic form of compound I of the invention.

FIG. 3 Shows the XRPD of a polymorphic form of compound I isolated from methanol.

FIG. 4 Shows the XRPD of the polymorphic form of compound I of the invention.

FIG. 5 shows the XRPD overlay of the polymorphic form of compound I of the invention (bottom trace) and the form isolated from methanol (top trace).

FIG. 6 shows the XRPD dissolution profile of 3 forms of compound I namely hydrate (far left) polymorph of the invention (far right) and form isolated from methanol (middle).

FIG. 7 shows the sorption desorption profile cycle 1 for compound 1 hydrate showing water sorption kinetics at 25° C.

FIG. 8 shows the water sorption/desorption isotherm cycle for compound 1 hydrate showing water sorption kinetics at 25° C.

FIG. 9 shows the sorption desorption profile cycle 1 for compound 1 anhydrate form of the invention showing water sorption kinetics at 25° C.

FIG. 10 shows the water sorption/desorption isotherm cycle for compound 1 anhydrate form of the invention showing water sorption kinetics at 25° C.

FIG. 11 shows the sorption desorption profile cycle 1 for compound 1 anhydrate form isolated from methanol showing water sorption kinetics at 25° C.

FIG. 12 shows the water sorption/desorption isotherm cycle for compound 1 anhydrate form isolated from methanol showing water sorption kinetics at 25° C.

### DETAILED DESCRIPTION

The applicants of the present application have now identified a polymorphic form of compound I that has acceptable dissolution properties whilst at the same time being the anhydrate form and can be readily handled and can be reproducibly incorporated in a pharmaceutical dosage form. In addition the water sorption profile of this polymorph was significantly lower than either the corresponding hydrate or the known polymorphic form

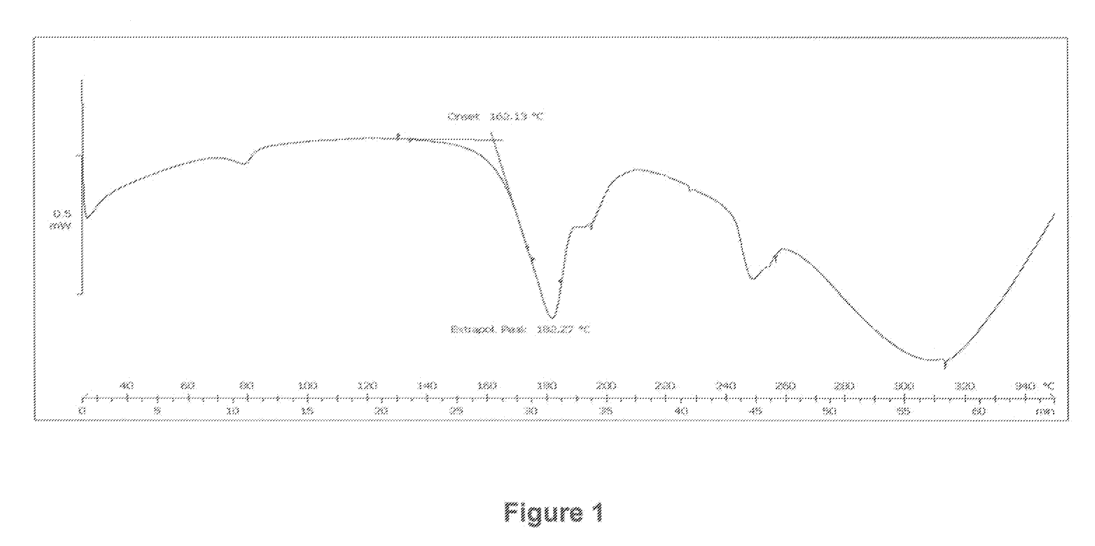

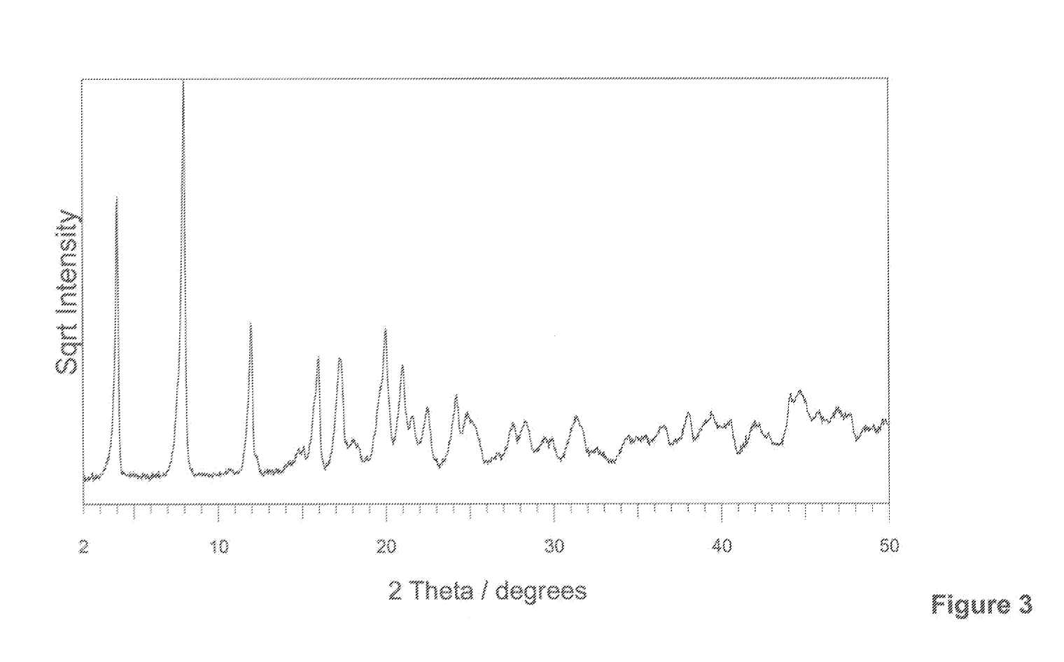

Initial studies into compound I involved analysis of the material isolated from the natural sources. In most instances these materials were isolated from the natural sources using methanol as an extractant and so the initial polymorphic form known was that isolated from methanol. A DSC of the material isolated from methanol is shown in FIG. 1 and the XRPD is shown in FIG. 3.

Unfortunately analysis of this polymorphic form indicated that it was somewhat hygroscopic in that it absorbed water which meant that it was difficult to handle in a manufacturing sense as in order to produce reproducible dosage levels there is a requirement for consistent levels of water to be present. As this polymorphic form absorbed water it was therefore difficult to formulate consistently.

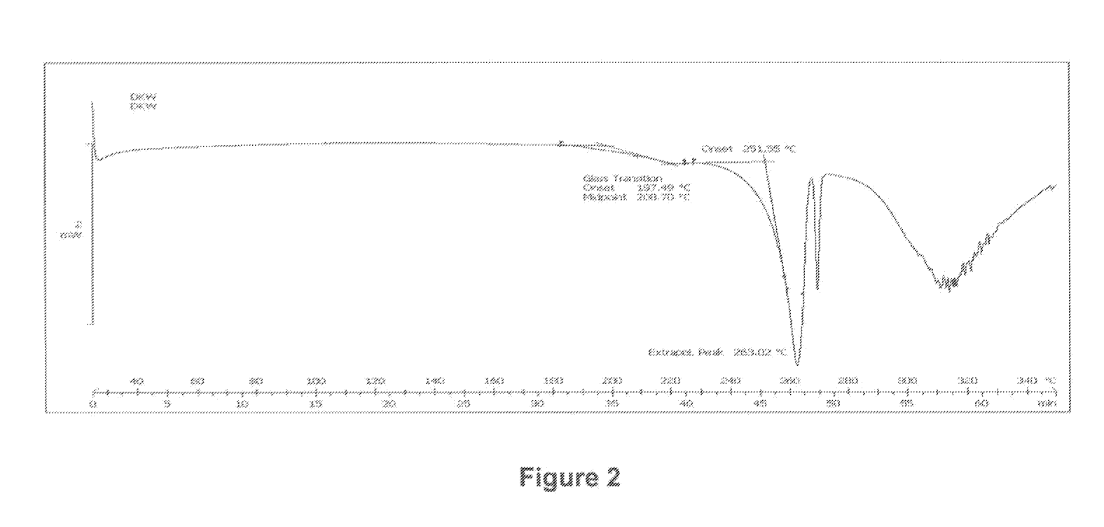

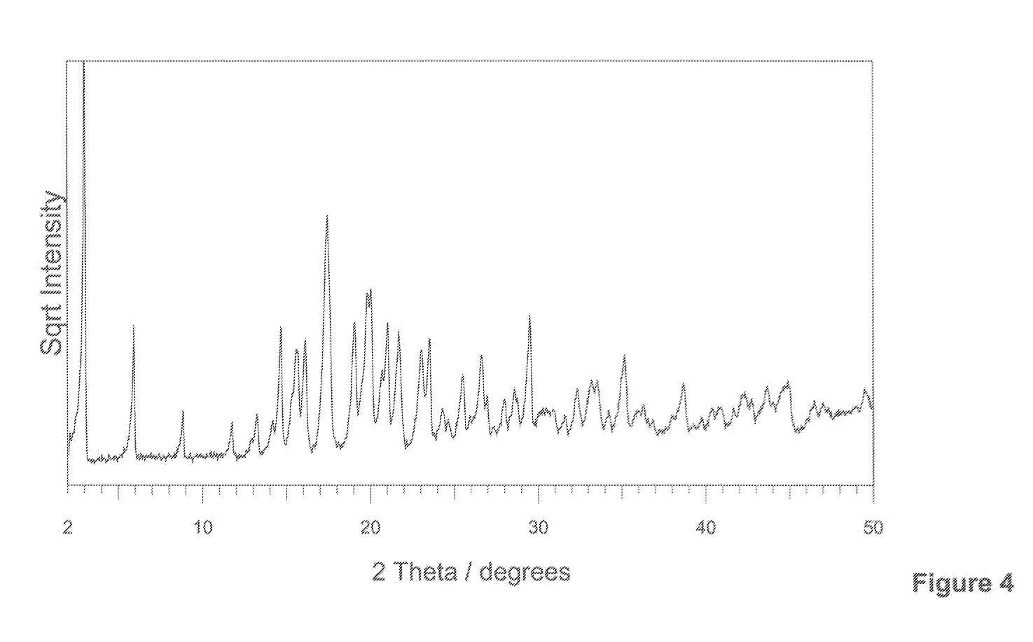

In the search for new polymorphic forms the applicant reslurried compound I at elevated temperature in isopropanol and identified a polymorphic form that was different to the polymorphic form identified from methanol. A copy of the DSC for this polymorphic form is shown in FIG. 2 and the XRPD is shown in FIG. 4.

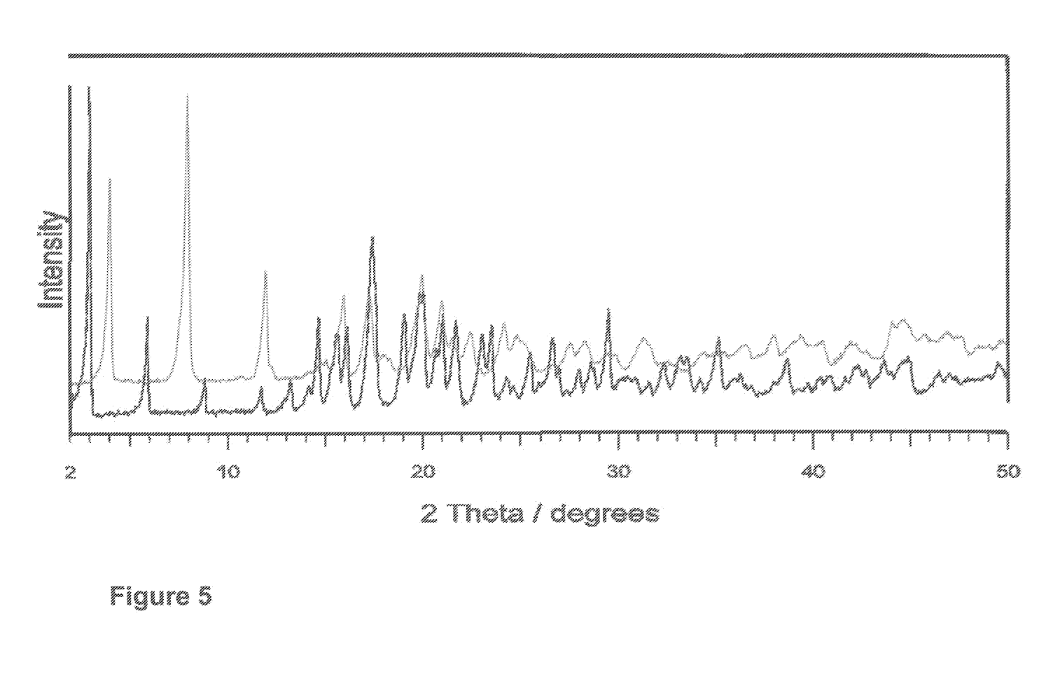

In addition overlaying the XRPD of the form isolated from methanol over the form from isopropanol (FIG. 5) clearly demonstrated that the two crystalline forms could be differentiated from each other.

Analysis of the XRPD of the polymorphic form of the invention allows the identification of the key X-ray diffraction peaks. A summary of the key peaks is given in table 1.

TABLE 1
List of significant X-ray powder diffraction (XRPD) peaks
the polymorphic form of compound I of the invention

| Position of Peak | |
|---|---|
| (2-theta°, ±0.02°) | Relative intensity |
| 2.96 | Strong |
| 5.88 | Weak |
| 14.66 | Weak |
| 15.57 | Weak |
| 15.64 | Weak |
| 16.12 | Weak |
| 17.33 | Medium |
| 17.43 | Medium |
| 17.60 | Medium |
| 19.06 | Weak |
| 19.84 | Medium |
| 20.03 | Medium |
| 21.02 | Weak |
| 21.71 | Weak |
| 23.55 | Weak |
| 29.53 | Weak |

As can be seen the crystalline form of the polymorph of compound I of the invention

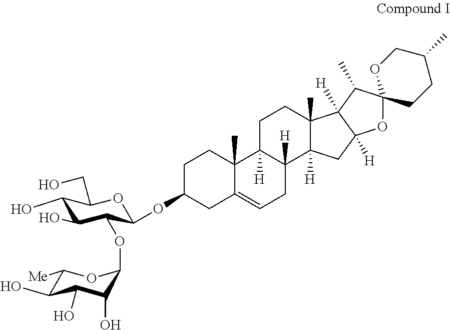

may be characterised as showing on X-ray diffraction a peak on the 2theta scale at 2.96±0.02°.

In some embodiments the crystalline form may be further characterised as showing on X-ray diffraction at least 1 peak on the 2theta scale selected from the group consisting of 17.33°±0.02°, 17.43°±0.02°, 17.60°±0.02°, 19.84°±0.02°, and 20.03°±0.02°.

In some embodiments the crystalline form may be further characterised as showing on X-ray diffraction at least 2 peaks on the 2theta scale selected from the group consisting of 17.33°±0.02°, 17.43°±0.02°, 17.60°±0.02°, 19.84°±0.02°, and 20.03°±0.02°.

In some embodiments the crystalline form may be further characterised as showing on X-ray diffraction at least 3 peaks on the 2theta scale selected from the group consisting of 17.33°±0.02°, 17.43°±0.02°, 17.60°±0.02°, 19.84°±0.02°, and 20.03°±0.02°.

In some embodiments the crystalline form may be further characterised as showing on X-ray diffraction at least 4 peaks on the 2theta scale selected from the group consisting of 17.33°±0.02°, 17.43°±0.02°, 17.60°±0.02°, 19.84°±0.02°, and 20.03°±0.02°.

In some embodiments the crystalline form may be further characterised as showing on X-ray peaks on the 2theta scale at 17.33°±0.02°, 17.43°±0.02°, 17.60°±0.02°, 19.84°±0.02°, and 20.03°±0.02°.

In some embodiments the crystalline form may be further characterised as showing on X-ray diffraction at least 1 peak on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

In some embodiments the crystalline form may be further characterised as showing on X-ray diffraction at least 4 peaks on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

In some embodiments the crystalline form may be further characterised as showing on X-ray diffraction at least 7 peaks on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

In some embodiments the crystalline form may be further characterised as showing on X-ray diffraction peaks on the 2theta scale at 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

As will be appreciated by a skilled worker in the field the relative intensities of the diffractions can vary depending upon a number of factors such as the method of the sample preparation and the type of instrument used. In addition in certain instances some of the peaks referred to above may not be detectable. Indeed the peaks listed above are merely the significant peaks as identified by the applicant. A complete listing of peaks (albeit small in many instances) is given in table 2.

TABLE 2
Complete list of X-ray diffraction peaks identified for
the polymorphic form of compound I of the invention

| Angle | d value | Intensity |
|---|---|---|
| (2-Theta) | (Angstrom) | Count |
| 2.96 ± 0.02 | 34.7 ± 0.2 | 1000 |
| 5.88 ± 0.02 | 17.45 ± 0.06 | 73.2 |
| 8.77 ± 0.02 | 11.69 ± 0.03 | 8.7 |
| 11.70 ± 0.02 | 8.77 ± 0.01 | 6.6 |
| 12.89 ± 0.02 | 7.97 ± 0.01 | 1.4 |
| 13.21 ± 0.02 | 7.78 ± 0.01 | 8.9 |
| 14.15 ± 0.02 | 7.26 ± 0.01 | 5.6 |
| 14.66 ± 0.02 | 7.01 ± 0.01 | 80.3 |
| 15.27 ± 0.02 | 6.731 ± 0.009 | 15 |
| 15.57 ± 0.02 | 6.605 ± 0.008 | 54.5 |
| 15.64 ± 0.02 | 6.574 ± 0.008 | 54.8 |
| 16.12 ± 0.02 | 6.382 ± 0.008 | 63.5 |
| 17.33 ± 0.02 | 5.937 ± 0.007 | 220.3 |
| 17.43 ± 0.02 | 5.905 ± 0.007 | 282.3 |
| 17.60 ± 0.02 | 5.846 ± 0.007 | 108.1 |
| 18.74 ± 0.02 | 5.493 ± 0.006 | 8.0 |
| 19.06 ± 0.02 | 5.404 ± 0.006 | 81.3 |
| 19.50 ± 0.02 | 5.281 ± 0.005 | 22.6 |
| 19.84 ± 0.02 | 5.193 ± 0.005 | 125.7 |
| 20.03 ± 0.02 | 5.144 ± 0.005 | 129.7 |
| 20.68 ± 0.02 | 4.982 ± 0.005 | 29.3 |
| 21.02 ± 0.02 | 4.903 ± 0.005 | 78.5 |
| 21.71 ± 0.02 | 4.750 ± 0.004 | 61.5 |
| 23.05 ± 0.02 | 4.477 ± 0.004 | 45.3 |
| 23.55 ± 0.02 | 4.384 ± 0.004 | 50.4 |
| 24.29 ± 0.02 | 4.251 ± 0.003 | 4.5 |
| 24.67 ± 0.02 | 4.188 ± 0.003 | 1.4 |
| 25.51 ± 0.02 | 4.051 ± 0.003 | 20.1 |
| 25.97 ± 0.02 | 3.981 ± 0.003 | 2.2 |
| 26.66 ± 0.02 | 3.880 ± 0.003 | 34.6 |
| 26.96 ± 0.02 | 3.837 ± 0.003 | 7.4 |
| 28.00 ± 0.02 | 3.698 ± 0.003 | 6.8 |
| 28.61 ± 0.02 | 3.620 ± 0.002 | 10.3 |
| 28.80 ± 0.02 | 3.597 ± 0.002 | 5.5 |
| 29.53 ± 0.02 | 3.510 ± 0.002 | 75.2 |
| 30.17 ± 0.02 | 3.438 ± 0.002 | 1.9 |
| 30.48 ± 0.02 | 3.403 ± 0.002 | 3.1 |
| 30.95 ± 0.02 | 3.352 ± 0.002 | 2.2 |
| 31.63 ± 0.02 | 3.282 ± 0.002 | 1.3 |
| 32.38 ± 0.02 | 3.208 ± 0.002 | 9.3 |
| 32.95 ± 0.02 | 3.154 ± 0.002 | 5.1 |
| 33.24 ± 0.02 | 3.128 ± 0.002 | 13.9 |
| 33.57 ± 0.02 | 3.097 ± 0.002 | 13.6 |
| 34.24 ± 0.02 | 3.039 ± 0.002 | 1.8 |
| 35.20 ± 0.02 | 2.958 ± 0.002 | 29.2 |
| 35.98 ± 0.02 | 2.897 ± 0.002 | 2.1 |
| 36.33 ± 0.02 | 2.869 ± 0.002 | 4.1 |
| 36.59 ± 0.02 | 2.850 ± 0.002 | 0.9 |
| 36.90 ± 0.02 | 2.827 ± 0.001 | 0.9 |
| 38.07 ± 0.02 | 2.743 ± 0.001 | 1.4 |
| 38.71 ± 0.02 | 2.699 ± 0.001 | 12.1 |
| 39.80 ± 0.02 | 2.628 ± 0.001 | 1 |
| 40.46 ± 0.02 | 2.587 ± 0.001 | 2.4 |
| 40.93 ± 0.02 | 2.558 ± 0.001 | 2.6 |
| 41.71 ± 0.02 | 2.513 ± 0.001 | 2.7 |
| 42.20 ± 0.02 | 2.485 ± 0.001 | 5.6 |
| 42.42 ± 0.02 | 2.473 ± 0.001 | 7 |
| 42.79 ± 0.02 | 2.452 ± 0.001 | 4.6 |
| 43.75 ± 0.02 | 2.401 ± 0.001 | 9.8 |
| 44.07 ± 0.02 | 2.384 ± 0.001 | 4.1 |
| 44.62 ± 0.02 | 2.357 ± 0.001 | 8.4 |
| 45.04 ± 0.02 | 2.3354 ± 0.001 | 10.3 |
| 46.57 ± 0.02 | 2.2629 ± 0.0009 | 4.6 |
| 47.10 ± 0.02 | 2.2386 ± 0.0009 | 2.8 |
| 47.38 ± 0.02 | 2.2263 ± 0.0009 | 2.2 |
| 48.08 ± 0.02 | 2.1959 ± 0.0009 | 1.2 |
| 48.50 ± 0.02 | 2.1779 ± 0.0008 | 1.1 |
| 49.01 ± 0.02 | 2.1566 ± 0.0008 | 1.7 |
| 49.56 ± 0.02 | 2.1341 ± 0.0008 | 6.3 |

Once again this is a comprehensive list of peaks identified by the applicant. Based on the relative intensities of many of the peaks a skilled addressee would appreciate that analysis of the same polymorphic form on another instrument by a different researcher may not identify all the minor peaks identified above and the peaks in the table are provided merely as a comprehensive listing. For the purposes of identification it is submitted that the peaks identified in table 1, especially the strong and medium peaks, are more characteristic of the presence of the polymorph of the invention.

The present invention will now be described with reference to the following non-limiting examples.

### Example 1

### Polymorphic Form Isolated from Methanol (Comparative Example)

The hydrated form of compound I (1.00 g) was dried by a twice-repeated strip from methanol:chloroform 2:1. It was then dissolved in refluxing methanol (50 mL) and slowly cooled (over several hours) and then stirred at ambient temperature. The product was collected by filtration and washed with methanol then dried in vacuo.

### Example 2 Polymorphic Form of Compound of the Invention

Compound I (48.0 g, hydrated form) was stirred in 2-propanol (638 mL) for 2 hours and then with stirring heated to 75° C. at 0.5° C./min (over 110 min) and then stirred at 75° C. for 2 hours. The slurry was then cooled to 20° C. at 0.3° C./min (over 183 min) to 20° C. and then stirred at 20° C. for 16 hours under nitrogen. The resulting slurry was filtered (Glass funnel P3) and the filter-cake washed with 2-propanol (200 mL). The product was dried in vacuo for 3 days at ambient temperature to give the polymorphic form of compound 1 of the invention.

### Example 3 Differential Scanning Calorimetry

DSC data was collected on a Mettler Toledo DSC1 system using standard STARe software. Samples were prepared by manually pressing the material into standard 25 microlitre aluminium pans and running a standard scan regime of 5 degree/min temperature rise with 50 mL/min headspace nitrogen purge gas flow. The instrument was calibrated using indium and tin reference standard melting points. Onset, peak and glass transition temperatures were determined graphically using the Mettler Toledo STARe software. The results of this analysis on the materials produced in examples 1 and 2 is shown in FIGS. 1 and 2 respectively.

### Example 4 X-Ray Diffraction Analysis

The sample powders were lightly ground in an agate hand mortar to disaggregate them and then packed in well-type sample holders. Analysis was carried out in a Philips PW1700 series automated powder diffractometer equipped with automatic divergence slit, 0.2 mm receiving slit, no anti-scatter slit, graphite diffracted beam monochromator and xenon-filled proportional counter. Radiation used was the cobalt K alpha envelope (wavelength ˜1.79 Å). Data was recorded from 2 degrees two-theta to 50 degrees two-theta at 0.04 degree intervals, counting for 1 second per point. Lists of peak positions and relative intensities are the result of applying a peak-picking algorithm to background-stripped data; the intensity values are relative to that of the largest peak for each sample and represent peak heights. Relative intensities less than 0.5 parts per thousand have been ignored.

The details of the data collection are:
Angular range: 2 to 50° 2θ
Step size: 0.04° 2θ
Collection time: 1 s.step⁻¹.

The results are shown for the material in example 1 and example 2 in FIGS. 3, 4 and as a combined trace in FIG. 5.

### Example 5

### Dissolution Analysis

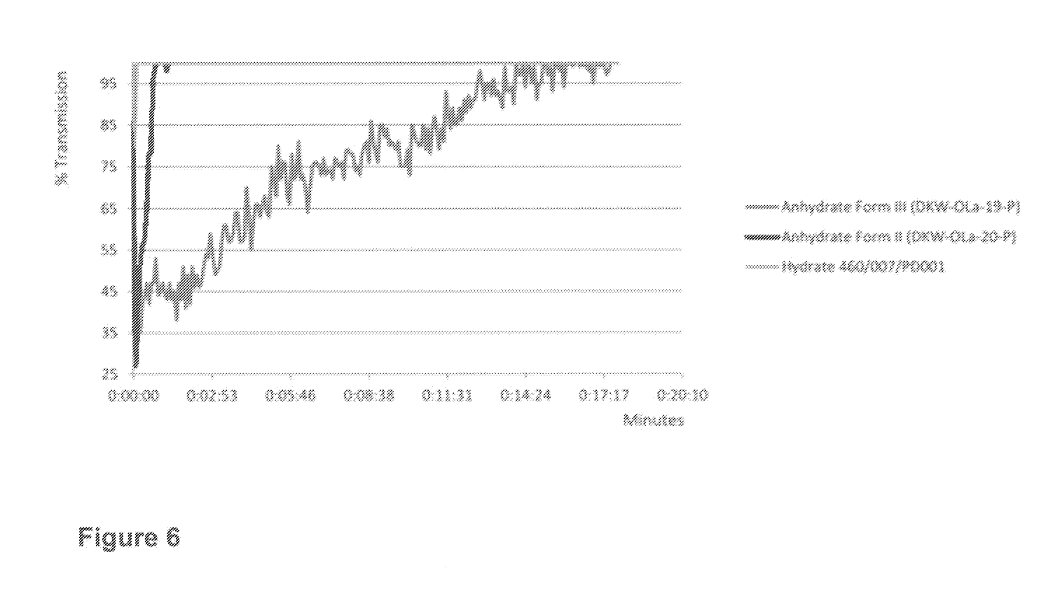

In order to test the solubility of the polymorphs produced in examples 1 and 2 a set amount of the material was added to ethanol with stirring and the % transmission of the mixture thus monitored until the % transmission reached 100% (which indicated complete dissolution). The 3 materials tested were (1) compound I hydrate, (2) the polymorphic form of compound I of the invention and (3) the polymorphic form of compound I isolated from methanol. The results of this experiment were detailed in FIG. 6. Whilst the hydrate dissolves the fastest the polymorphic form isolated from methanol has a dissolution rate that is still very rapid almost being comparable to the dissolution profile of the hydrate. In contrast the dissolution profile of the polymorphic form of the invention is much slower taking almost 8 times as long to reach full dissolution. This suggests that this polymorphic form is significantly different and is likely to be the most stable over time.

### Example 6

### Bulk Density and Tap Density

A comparative study of the bulk density and the tapped density of the 3 solid forms of compound one were carried out. The 3 materials tested were (1) compound I hydrate, (2) the polymorphic form of compound I of the invention and (3) the polymorphic form of compound I isolated from methanol. The tests indicated the weight of material in 5.0 cm³ of solid (g) to calculate the bulk density. This was then tapped 50 times and the tapped density calculated. The results are shown in Table 3.

| | Weight of 5.0 | | Volume after | Tapped |
|---|---|---|---|---|
| | cm³ of Solid | Bulk density | tapped 50 | density |
| Form | (g) | (g/cm³) | times (cm³) | (g/cm³) |
| 1 | 1.430 | 0.29 | 3.9 | 0.37 |
| 2 | 1.489 | 0.30 | 3.6 | 0.41 |
| 3 | 0.847 | 0.17 | 3.2 | 0.26 |

1 compound I hydrate,
2 the polymorphic form of compound I of the invention and
3 the polymorphic form of compound I isolated from methanol.

As can be seen the polymorphic form of the invention had the highest bulk and tapped density. From a transportation perspective this is particularly attractive as this polymorphic form can be efficiently transported.

### Example 7

### Dynamic Vapour Sorption (DVS)

A comparative study of the dynamic vapour sorption (DVS) of the 3 solid forms of compound one were carried out. The 3 materials tested were (1) compound I hydrate, (2) the polymorphic form of compound I of the invention and (3) the polymorphic form of compound I isolated from methanol. The samples were analysed on a DVS automated moisture sorption instrument at 25° C. with sample sizes of 25-52 mg for the analysis. The samples were initially dried for 300 minutes under a continuous flow of air to establish the dry mass. The samples were then exposed to the following typical partial pressure profile: 0% to 90% RH in 10% steps and then followed by a 5% step to 95%. The partial pressure was then decreased in a similar manner.

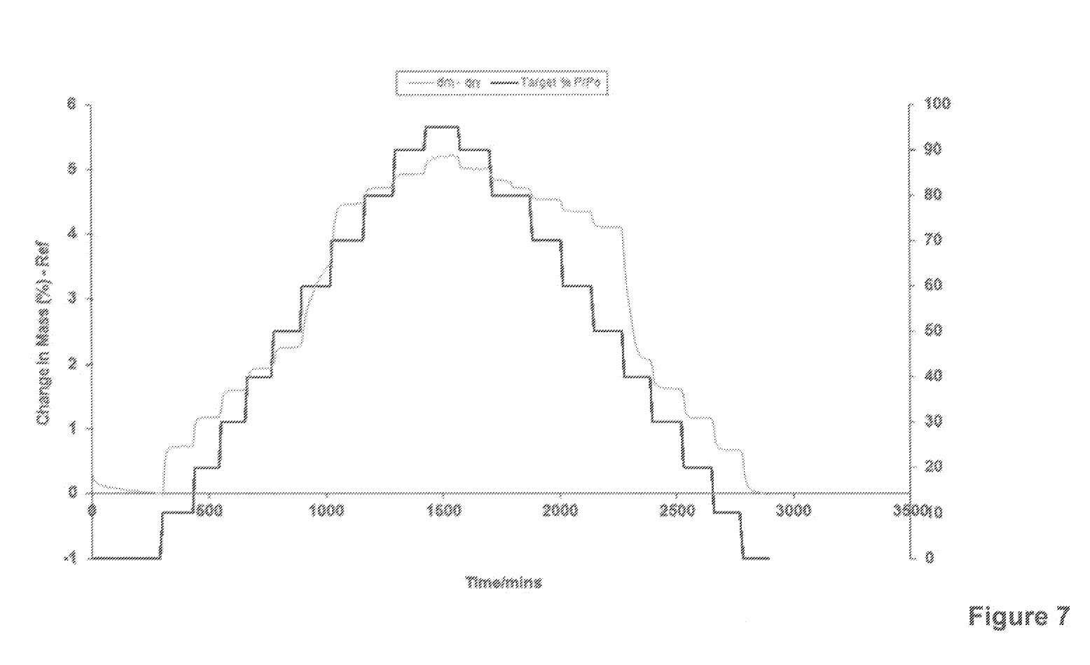

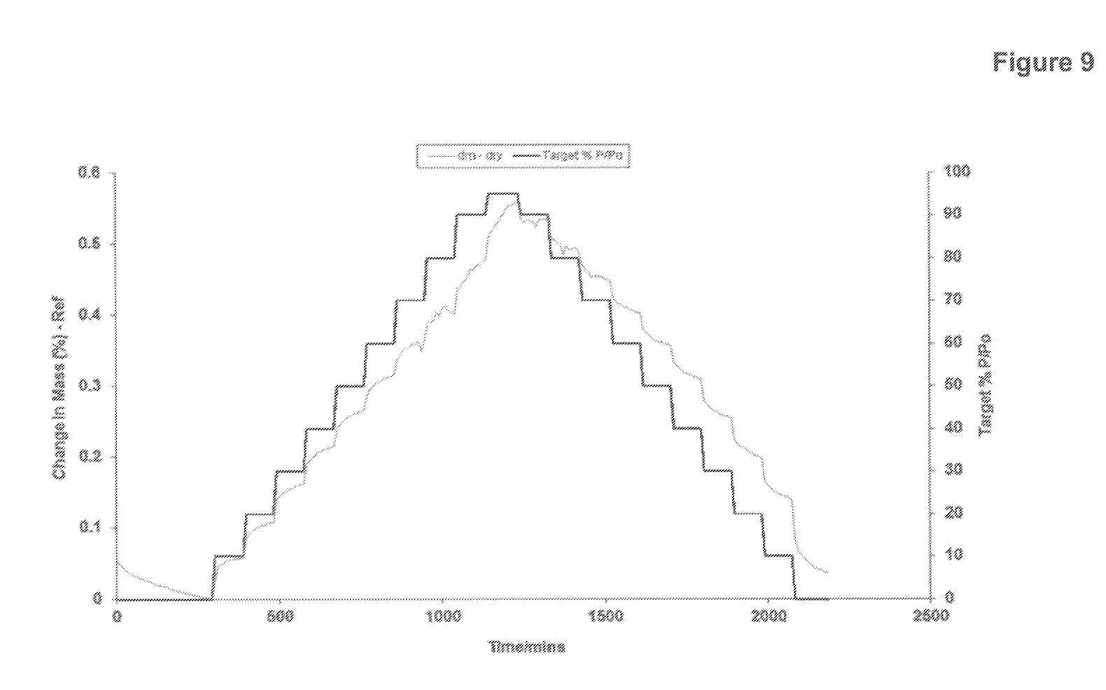

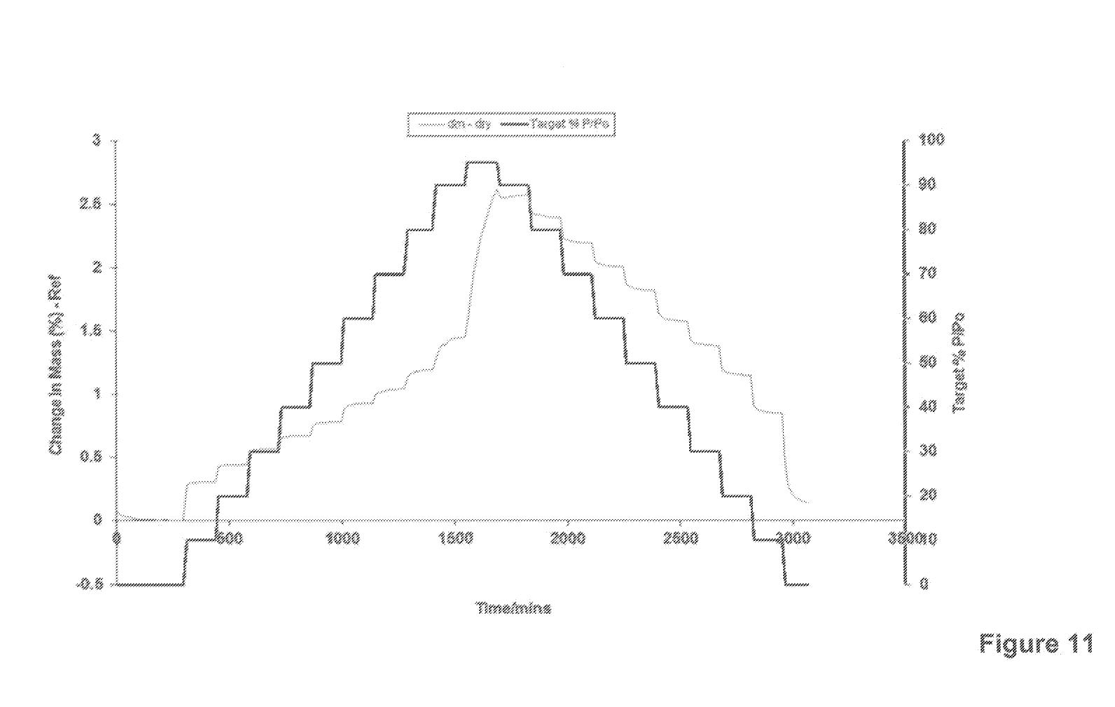

Typical net percent change in mass (based on dry mass) versus time plots for the first cycle at 25° C. for the three samples are shown in FIGS. 7, 9 and 11. FIG. 7 shows the mass plot for the hydrate, FIG. 9 shows the mass plot for the sample polymorphic form of the invention and FIG. 11 shows the mass plot for the polymorphic form isolated from methanol. The line plotted on the left y-axis, indicates the percentage change in mass referenced to the dry mass (after initial drying stage), m₀, as a function of time. The other line, plotted on the right y-axis, traces the requested % partial pressure of water vapour in the DVS as a function of time.

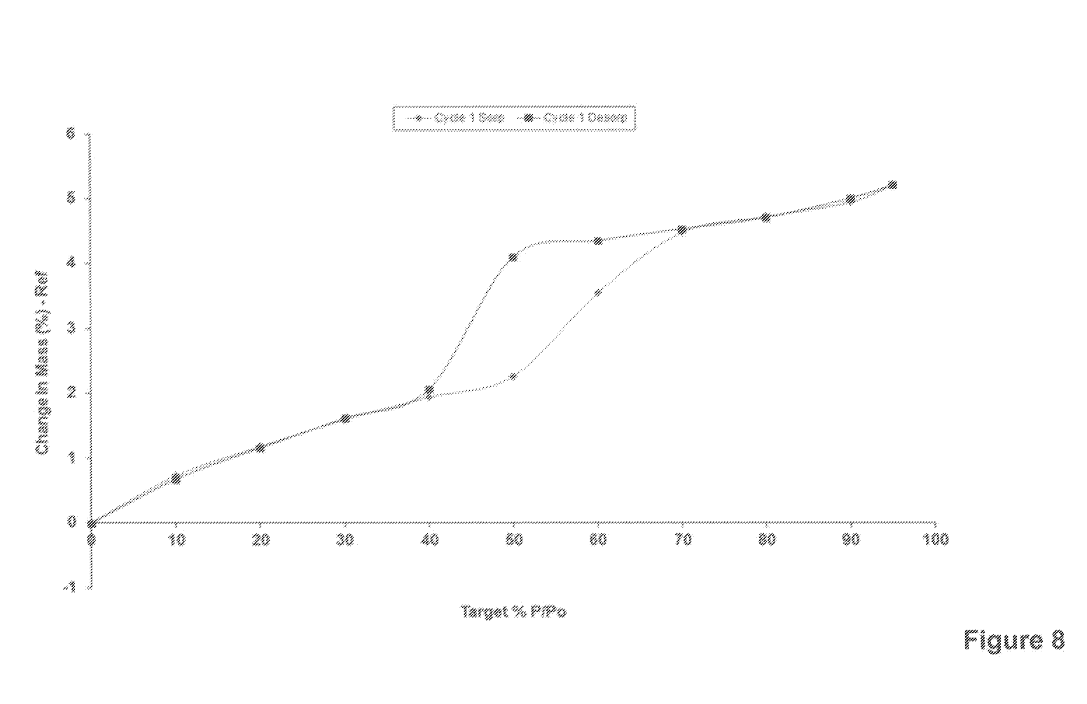

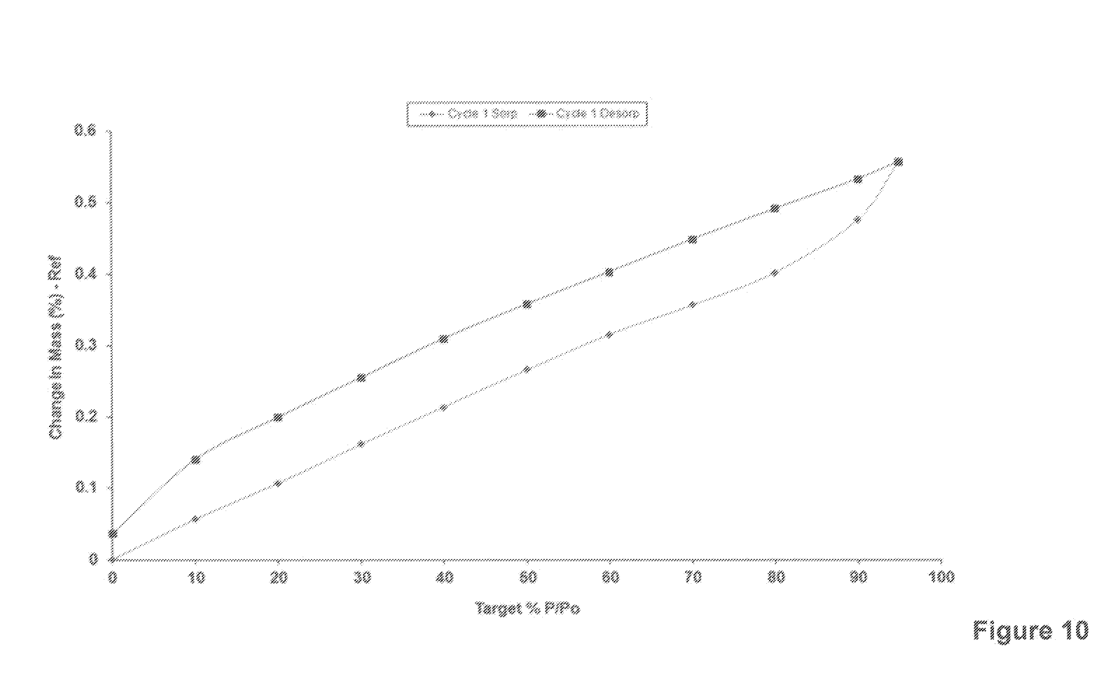

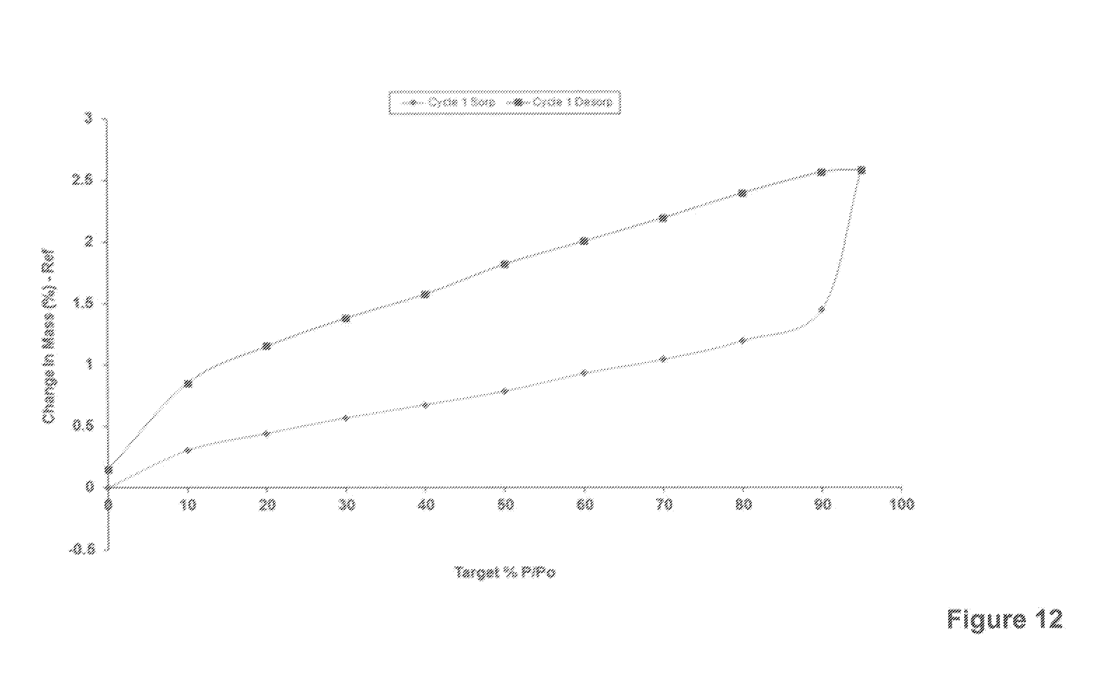

The water vapour sorption isotherm plots for the three samples at 25° C. are shown in FIGS. 8, 10 and 12. FIG. 8 shows the isotherm plot for the hydrate, FIG. 10 shows the isotherm plot for the polymorphic form of the invention and FIG. 12 shows the isotherm plot for the polymorphic form isolated from methanol. The isotherm plots display the percent change in mass (referenced from the dry mass, m₀) versus the requested relative humidity.

For all the RH steps, the instrument was run in a dm/dt mode (mass variation over time variation). A fixed dm/dt value of 0.002% min-1 was selected. This criterion permits the DVS software to automatically determine when equilibrium has been reached and complete a relative humidity step. When the rate of change of mass falls below this threshold over a determined period of time, the humidity will proceed to the next programmed level. A maximum stage time of 360 minutes and a minimum stage time of 10 minutes were selected for this experiment.

The water vapour sorption results for the samples at 25° C. (FIGS. 7, 9 and 11), indicate the three samples exhibit different water vapour sorption characteristics and there are measurable differences in water uptake between the samples. The percentage of water uptake for the hydrate and the polymorph obtained from methanol is relatively high, indicating bulk absorption. The total moisture uptake for the anhydrate is about 5.2% and for the polymorph from methanol is about 2.5%. In contrast for the polymorphic form of the invention the percentage of water uptake is low, and is about 0.55%, indicating surface absorption. Accordingly the polymorphic form of the invention is less hygroscopic than either the hydrate or the known polymorphic form.

The details of specific embodiments described in this invention are not to be construed as limitations. Various equivalents and modifications may be made without departing from the essence and scope of this invention, and it is understood that such equivalent embodiments are part of this invention.

1. A crystalline form of a compound of the formula: 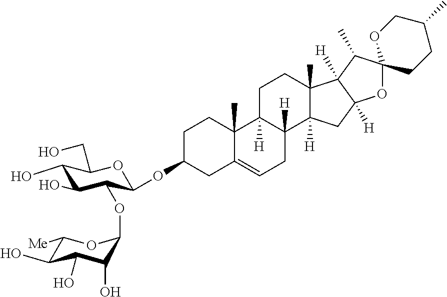
which shows on X-ray diffraction a peak on the 2theta scale at 2.96±0.02°.

2. A crystalline form according to claim 1 which also shows on X-ray diffraction at least 1 peak on the 2theta scale selected from the group consisting of 17.33°±0.02°, 17.43°±0.02°, 17.60°±0.02°, 19.84°±0.02°, and 20.03°±0.02°.

3. A crystalline form according to claim 1 which also shows on X-ray diffraction at least 2 peaks on the 2theta scale selected from the group consisting of 17.33°±0.02°, 17.43°±0.02°, 17.60°±0.02°, 19.84°±0.02°, and 20.03°±0.02°.

4. A crystalline form according to claim 1 which also shows on X-ray diffraction at least 3 peaks on the 2theta scale selected from the group consisting of 17.33°±0.02°, 17.43°±0.02°, 17.60°±0.02°, 19.84°±0.02°, and 20.03°±0.02°.

5. A crystalline form according to claim 1 which also shows on X-ray diffraction at least 4 peaks on the 2theta scale selected from the group consisting of 17.33°±0.02°, 17.43°±0.02°, 17.60°±0.02°, 19.84°±0.02°, and 20.03°±0.02°.

6. A crystalline form according to claim 1 which also shows on X-ray peaks on the 2theta scale at 17.33°±0.02°, 17.43°±0.02°, 17.60°±0.02°, 19.84°±0.02°, and 20.03°±0.02°.

7. A crystalline form according to claim 1 which shows on X-ray diffraction at least 1 peak on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

8. A crystalline form according to claim 1 which shows on X-ray diffraction at least 4 peaks on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

9. A crystalline form according to claim 1 which shows on X-ray diffraction at least 7 peaks on the 2theta scale selected from the group consisting of 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

10. A crystalline form according to claim 1 which shows on X-ray diffraction peaks on the 2theta scale at 5.88°±0.02°, 14.66°±0.02°, 15.57°±0.02°, 15.64°±0.02°, 16.12°±0.02°, 19.06°±0.02°, 21.02°±0.02°, 21.71°±0.02°, 23.55°±0.02°, and 29.53°±0.02°.

11. A pharmaceutical composition comprising a crystalline form according to claim 1.
